(19) United States
(12) Patent Application Publication
(10) Pub. No.: US 2009/0257964 A1
(43) Pub. Date: Oct. 15, 2009

(54) Fluoride-releasing strips for caries prevention

(75) Inventors: Bor-Shiunn Lee, Taipei (TW); Da-Ming Wang, Taipei City (TW); Juin-Yih Lai, Taoyuan County (TW); Chun-Pin Lin, Taipei (TW); Chun-Yu Chen, Taipei City (TW); Fang-Yu Liu, Taipei City (TW); Hao-Ting Wang, Taipei City (TW)

Correspondence Address: WPAT, PC, 7225 BEVERLY ST., ANNANDALE, VA, 22003, US

(73) Assignee: NATIONAL TAIWAN UNIVERSITY, Taipei City (TW)

(21) Appl. No.: 12/487,896

(22) Filed: Jun. 19, 2009

Related U.S. Application Data
(63) Continuation-in-part of application No. 11/760,894, filed on Jun. 11, 2007.

(51) Int. Cl. A61K 8/21; A61P 1/02

(52) U.S. Cl. 424/52

(57) ABSTRACT

The present invention discloses fluoride-releasing strips for caries prevention, wherein the fluoride-releasing strips comprises a colloidal fluoride-containing solution for releasing fluorine ion and a support substrate. A colloidal fluoride-containing solution comprises a fluoride solution, at least one buffer, at least one moisturizer and a carboxymethyl cellulose (CMC). The concentration of fluoride ions in said colloidal fluoride-containing solution ranges from 2500 ppm to ppm. The support substrate is waterproof material and the colloidal fluoride-containing solution is applied on the support substrate.

### CROSS REFERENCE TO RELATED APPLICATIONS

This application is a continuation in part of applicant's earlier application, Ser. No. 11/760,894, filed Jun. 11, 2007.

### BACKGROUND OF THE INVENTION

1. Field of the Invention

The present invention is generally related to a strip, and more particularly to a fluoride-releasing strip for caries prevention.

2. Description of the Prior Art

Oral healthiness is important to the state of human health. In addition, chewing malfunction will result in insufficient absorption of nutrients and thereby affect the healthiness of the whole body. Among a variety of reasons affecting oral healthiness, dental caries is accounted for an important one. For example, dental restoration, endodontic treatment, prosthetic tooth fabrication, and tooth extraction almost all result from dental caries. Therefore, most of the medical treatment resources for maintaining oral healthiness are used to cure the follow-up problems of dental caries.

The concentration of fluoride in the traditional fluoride gel are equal to (or more than) 12300 ppm. Fluoride is a toxic ion, so that it is dangerous for patient such as young children who may swallow the fluoride gel. The more fluoride it contains, the more fluoride the children may swallow.

In light of the above mentioned matter, in order to prevent dental caries, the effectiveness of these fluoride-containing products and the safety and convenience in the use of these fluoride-containing products are important factors, besides having good personal tooth cleaning habit. Providing a simple fluoride-releasing strip for caries prevention becomes an important research subject.

### SUMMARY OF THE INVENTION

In light of the above background, in order to fulfill the requirements of the industry, the invention provides a fluoride-releasing strip for caries prevention.

One object of the present invention is providing a fluoride-releasing strip for caries prevention. The fluoride-releasing strip for caries prevention comprises a colloidal fluoride-containing solution for releasing fluorine ions and a support substrate. The colloidal fluoride-containing solution is applied on the support substrate. The concentration of fluoride ions in the colloidal fluoride-containing solution ranges from 2500 ppm to 4600 ppm. Therefore, the fluoride-releasing strip is safer than the traditional fluoride gel.

Another object of the present invention is forming calcium fluoride (CaF₂) on the tooth. After present invention fluoride-releasing strip contacts with tooth, the calcium fluoride (CaF₂) is formed on the surface of tooth.

Another object of the present invention is providing a fluoride-releasing strip for caries prevention for short-time (less than 6 hours) or long-time (equal or more than 6 hours) use.

### BRIEF DESCRIPTION OF THE DRAWINGS

FIG. 1 is the Raman spectrometer. Using the strips for 0.5 hr, CaF₂ with characteristic peak at 322 nm is formed on the tooth.

FIG. 2 shows the result of demineralization inhibition test for 0, 4, 8, 24 hrs. Compared with the control group without using strips, use of strips for 4, 8, or 24 hrs can significantly inhibit caries progression.

FIG. 3 shows the result of demineralization inhibition test for 0, 1, 2, 4 hrs. Compared with the control group without using strips, use of strips for 1, 2, or 4 hrs can significantly inhibit caries progression.

FIG. 4 shows the result of demineralization inhibition test for 0, 0.5, 1 hr. Compared with the control group without using strips, use of strips for 0.5 or 1 hr can significantly inhibit caries progression.

FIG. 5 shows the result of demineralization inhibition test for 0, 7.5, 15, 30 mins. Compared with the control group without using strips, use of strips for 7.5, 15, 30 mins can significantly inhibit caries progression.

FIG. 6 shows the result of demineralization inhibition test of strips (CMC) compared with high concentration fluoride gel (APF, 12300 ppm). The effect of inhibiting caries progression is not different for using strips or APF. Both materials are applied for 4 minutes.

FIG. 7 shows the result of remineralization-promotion test for 0, 2, 4, 8 hrs. Compared with the control group without using strips, use of strips for 2, 4, or 8 hrs can significantly enhance tooth remineralization.

FIG. 8 is the positive model of Secondary Ion Mass Spectrometry (SIMS). Using the strips on the tooth (dash line), the CaF₂ is formed. CaF₂ shows the characteristic peak at 59 m/z.

FIG. 9 is the negative model of SIMS. Using the strips on the tooth, the fluoride concentration on the surface of sample increases (the peak at 19 m/z).

FIG. 10 shows the relationship of the fluoride concentration on the enamel surface and fluoride-containing strip tack time (n=15).

### DESCRIPTION OF THE PREFERRED EMBODIMENTS

What is probed into the invention is a fluoride-releasing strip for caries prevention. Detail descriptions of the processes and composition structures will be provided in the following in order to make the invention thoroughly understood. For those who are skilled in the art, generally the weight ratio is used to represent the ratio of each composition in the formula. Therefore, in the following description of the invention, the weight ratio is used to represent the ratio of each composition in the formula. Obviously, the application of the invention is not confined to specific details familiar to those who are skilled in the art. On the other hand, the compositions and processes that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In one embodiment of the present invention, a fluoride-releasing strip for caries prevention is provided. The fluoride-releasing strip for caries prevention comprises a colloidal fluoride-containing solution for releasing fluorine ions and a support substrate. The colloidal fluoride-containing solution is applied on the support substrate. When using the fluoride-containing strip, one hand contacts with the support substrate directly in order to handle the fluoride-containing strip. Then, the fluoride-containing surface is contacted with teeth. It is very convenient to use it. The above mentioned colloidal fluoride-containing solution comprises: a fluoride solution, at least one buffer, at least one moisturizer for inhibiting water dissipation and a carboxymethyl cellulose (CMC). The concentration of fluoride ions in the colloidal fluoride-containing solution ranges from 2500 ppm to 4600 ppm.

The fluoride-releasing strip for caries prevention is contacted with tooth for more than (or equal to) 6 hours. Calcium fluoride (CaF₂) is formed on the surface of tooth. The demineralization of the tooth contacting with said fluoride-releasing strip for 6 hours is less than 0.7 mg/cm².

The fluoride solution comprise one selected from a group consisting of the following: sodium fluoride (NaF), sodium monofluorophosphate(Na₂FPO₃), and stannous fluoride(SnF₂). The preferred fluoride solution is sodium fluoride (NaF) solution.

The buffer is used to adjust the pH value of said colloidal fluoride-containing solution. The viscosity of the mixture is increased gradually. The first range is from 6.2 to 6.5 and the pH value of the fluoride-containing solution is neutral. The second range is about 4˜5. When the pH value of the fluoride-containing solution is adjusted to around 4˜5, teeth may be slightly eroded to have micropores on the surface thereof so as to have fluorides enter the micropores and to thereby release fluoride ion for a longer period of time. Thus, the dental caries prevention can be promoted. In addition, the composition of the buffer comprises one compound selected from the group consisting of the following or any combination thereof: phosphates, nitrates, acetates, nitrites, borates, and oxalates.

In this embodiment, the composition of the moisturizer comprises one compound selected from the group consisting of the following or any combination thereof: glycerin, butylene glycol, hyaluronic acid, ceramides, hyaluronic acid, and collagen. But, it is not limited to the above examples. A preferred example according to the invention, the moisturizer is glycerin with concentration of 3.0 g/100 ml H₂O to 5.0 g/100 ml H₂O.

In this embodiment, the concentration of said carboxymethyl cellulose ranges from 2 to 2.5 wt %.

In this embodiment, the support substrate is waterproof material. Preferably, the support substrate comprises one substance selected from the group consisting of the following: handi-wrap, waterproof non-woven fabrics, and plastic films.

In another embodiment of the present invention, a fluoride-releasing strip for caries prevention is provided. The fluoride-releasing strip for caries prevention comprises a colloidal fluoride-containing solution for releasing fluorine ions and a support substrate. The colloidal fluoride-containing solution is applied on the support substrate. When using the fluoride-containing strip, one hand contacts with the support substrate directly in order to handle the fluoride-containing strip. Then, the fluoride-containing surface is contacted with teeth. It is very convenient to use it. The above mentioned colloidal fluoride-containing solution comprises: a fluoride solution, at least one buffer, at least one moisturizer for inhibiting water dissipation and a carboxymethyl cellulose (CMC). The concentration of fluoride ions in the colloidal fluoride-containing solution ranges from 2500 ppm to 4600 ppm.

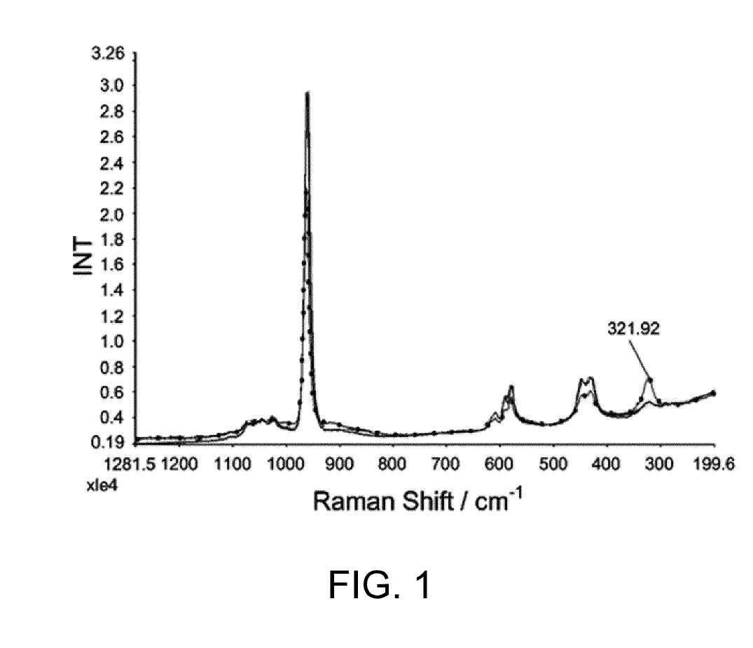

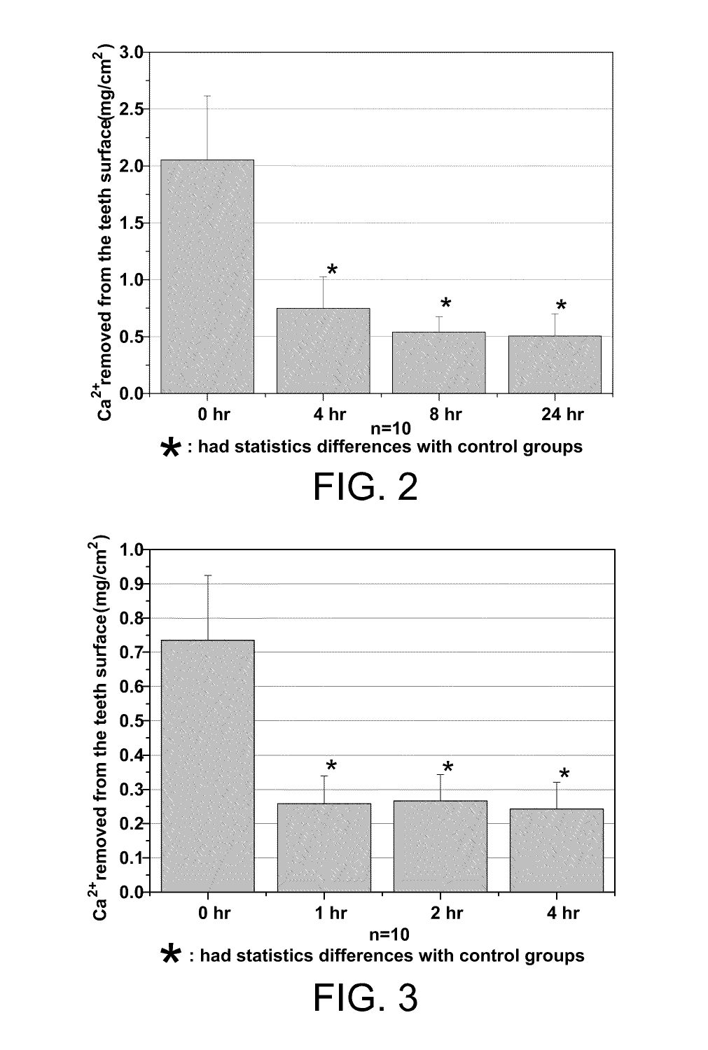

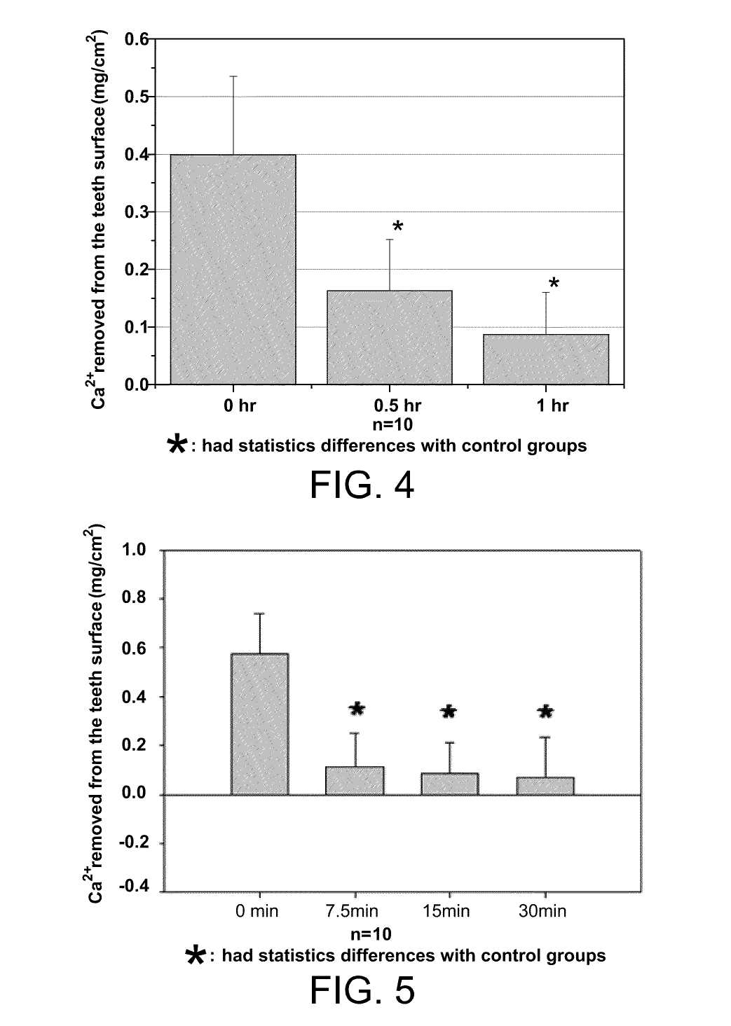

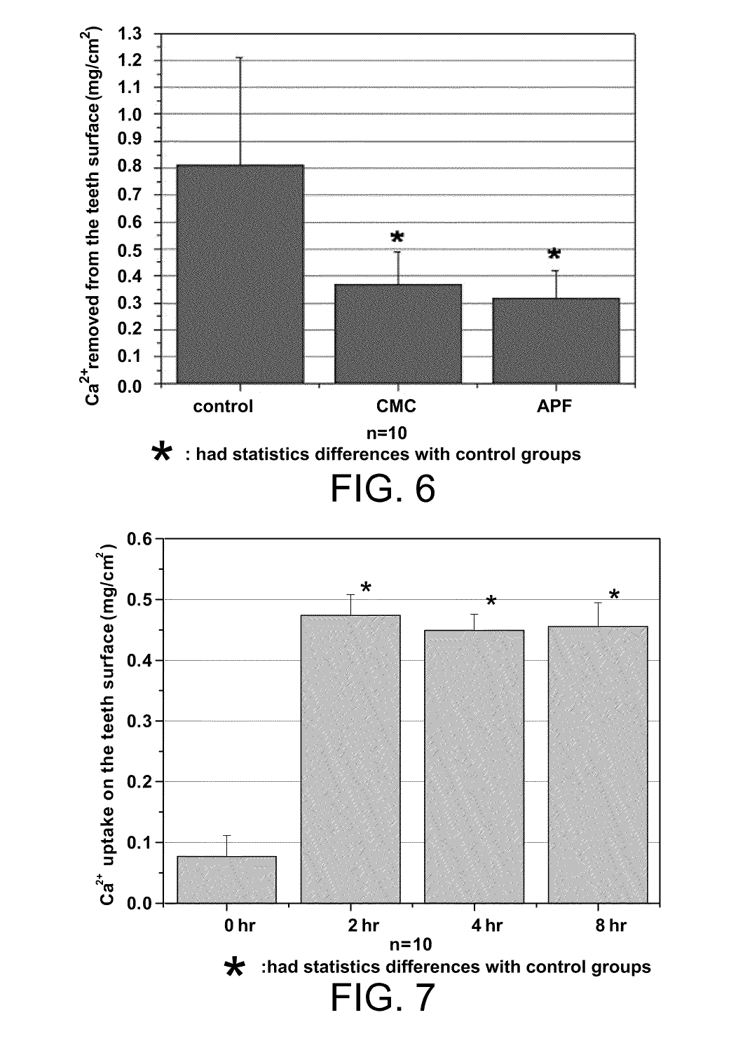

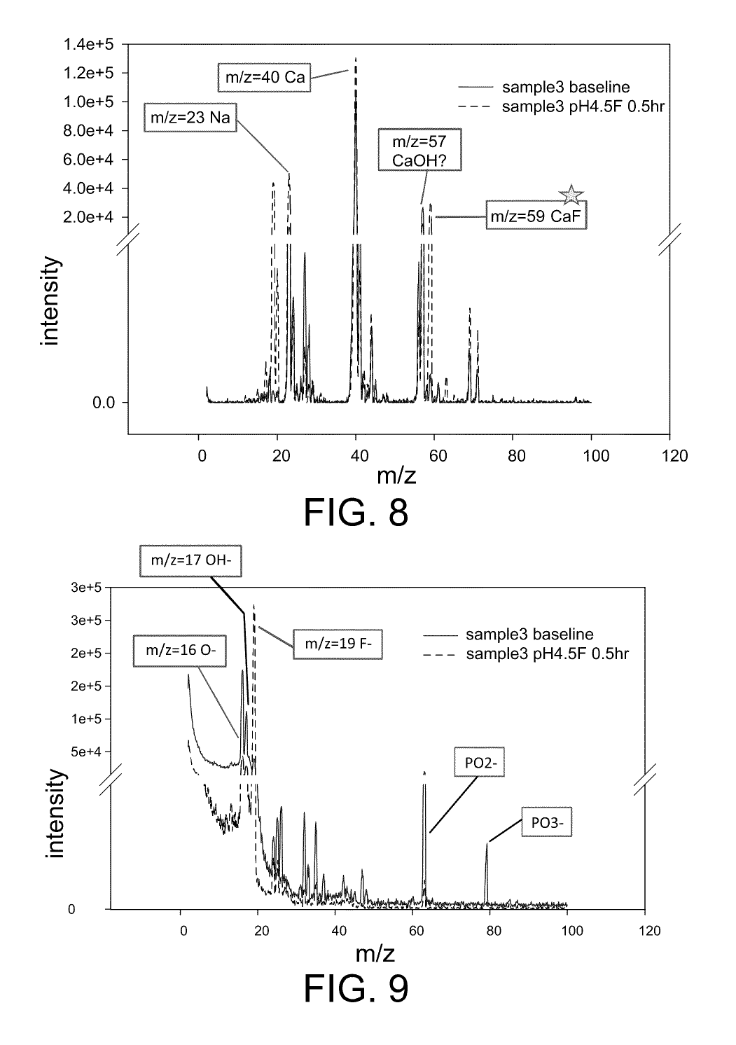

The preferred fluoride-releasing strip for caries prevention may be contacted with tooth less than 24 hours. Patient can use it for less than 0.5 hour (such as 1 min, 10 min) (FIG. 4, 5), or patient can use it for equal to (or more than) 0.5 hour (such as 1 hour, over night) (FIG. 2, 3). Calcium fluoride (CaF₂) is formed on the surface of tooth (FIG. 1, 8, 9). The demineralization of the tooth contacting with said fluoride-releasing strip for 6 hours is less than 0.7 mg/cm².

The fluoride solution comprise one selected from a group consisting of the following: sodium fluoride (NaF), sodium monofluorophosphate (Na₂FPO₃), and stannous fluoride (SnF₂). The preferred fluoride solution is sodium fluoride (NaF) solution.

The buffer is used to adjust the pH value of said colloidal fluoride-containing solution. The viscosity of the mixture is increased gradually. The first range is from 6.2 to 6.5 and the pH value of the fluoride-containing solution is neutral. The second range is about 4˜5. When the pH value of the fluoride-containing solution is adjusted to around 4˜5, teeth may be slightly eroded to have micropores on the surface thereof so as to have fluorides enter the micropores and to thereby release fluoride ion for a longer period of time. Thus, the dental caries prevention can be promoted. In addition, the composition of the buffer comprises one compound selected from the group consisting of the following or any combination thereof: phosphates, nitrates, acetates, nitrites, borates, and oxalates.

In this embodiment, the composition of the moisturizer comprises one compound selected from the group consisting of the following or any combination thereof: glycerin, butylene glycol, hyaluronic acid, ceramides, hyaluronic acid, and collagen. But, it is not limited to the above examples. A preferred example according to the invention, the moisturizer is glycerin with concentration of is from 3.0 g/100 ml H₂O to 5.0 g/100 ml H₂O.

In this embodiment, the concentration of said carboxymethyl cellulose ranges from 2 to 2.5 wt %.

In this embodiment, the support substrate is a waterproof material. Preferably, the support substrate comprises one substance selected from the group consisting of the following: handi-wrap, waterproof non-woven fabrics, and plastic films.

In another embodiment of the present invention, a fluoride-releasing strip for tooth is provided. The fluoride-releasing strip for tooth comprises a fluoride-containing solution for releasing fluorine ions and a support substrate. The fluoride-containing solution is applied on the support substrate. When using the fluoride-containing strip, one hand contacts the support substrate directly in order to handle the fluoride-containing strip and has the fluoride-containing surface face and contacted with teeth. It is very convenient for use. One formula for the fluoride-containing solution comprises a fluoride solution, at least one buffer, at least one moisturizer for inhibiting water dissipation, and a tackiness agent. The fluoride-ion concentration in the fluoride solution is about more than or equal to 1000 ppm. When the fluoride ions are from sodium fluoride solution, the fluoride-ion concentration is about 0.2 g NaF/100 ml H₂O. In this embodiment, the support substrate is waterproof material. Preferably, the support substrate comprises one substance selected from the group consisting of the following: handi-wrap, waterproof non-woven fabrics, and plastic films.

The buffer is used to adjust the pH value of the fluoride-containing solution. In the invention, adjusting the pH value directly by the addition of alkaline substance has the many disadvantages, such as causing local viscosity to rise drastically and producing precipitation. When the pH value is raised up to 4, condensation occurs. Therefore, the invention uses the buffer to adjust pH value. The viscosity of the mixture is thus increased gradually. The original pH value of the mixture is about 2.5˜3.5. The pH value of the fluoride-containing solution can be adjusted to become weak acidic or neutral. In this embodiment, the preferred pH value of the fluoride-containing solution has two ranges. The first range is 6.2˜6.5 and the pH value of the fluoride-containing solution is neutral. The second range is about 4˜5. When the pH value of the fluoride-containing solution is adjusted to around 4˜5, teeth may be slightly eroded to have micropores on the surface thereof so as to have fluorides enter the micropores and to thereby release fluoride ion for a longer period of time. Thus, the dental caries prevention can be promoted. In addition, the composition of the buffer comprises one compound selected from the group consisting of the following or any combination thereof: phosphates, nitrates, acetates, nitrites, borates, and oxalates.

In this embodiment, the composition of the moisturizer comprises one compound selected from the group consisting of the following or any combination thereof: glycerin, butylene glycol, hyaluronic acid, ceramides, hyaluronic acid, and collagen. But, it is not limited to the above examples. A preferred example according to the invention, the moisturizer is glycerin with concentration of 5.0 g/100 ml H₂O.

The preferred material for the tackiness agent is polyacrylic acid, such as a commercial product called carbopol. According to the invention, the preferred concentration for carbopol is between 2.5 g/100 ml H₂O and 5.0 g/100 ml H₂O. More preferably is 3 g/100 ml H₂O. When the concentration is more than 4 g carbopol/100 ml H₂O, the efficiency of the mass transfer of fluoride substance is reduced.

In another embodiment of the present invention, a method for forming the fluoride-containing solution is disclosed. At first, a fluoride solution is provided, where the fluoride-ion concentration of the fluoride solution is about more than or equal to 1000 ppm. At least one buffer is then added to the fluoride solution to form a first mixture solution. A moisturizer is then mixed with the first mixture solution to form a second mixture solution. Finally, a tackiness agent is added to the second mixture solution and then the second mixture solution is blended to form a colloidal fluoride-containing solution.

In another embodiment of the present invention, a method for forming the fluoride-containing solution is disclosed. At first, a sodium fluoride (NaF) solution is provided where the fluoride-ion concentration of the sodium fluoride (NaF) solution is about more than or equal to 1000 ppm. Next, at least one buffer is added to the fluoride solution to form a first mixture solution where the buffer comprises KH₂PO₄ and K₂HPO₄ with a molar ratio of 2:3. A moisturizer, such as glycerin, is then added and mixed with the first mixture solution to form a second mixture solution. The concentration of the moisturizer is about 5.0 g/100 ml H₂O (relative to the concentration of the final fluoride-containing solution). A tackiness agent, such as polyacrylic acid, is added to the second mixture solution to form a third mixture solution. The concentration of the tackiness agent is about 3.0 g/100 ml H₂O (relative to the concentration of the final fluoride-containing solution). Then, the third mixture solution is blended for about 6˜8 hrs until the third mixture solution becomes colloidal to form the fluoride-containing solution.

### EXAMPLE 1

The experiment uses a molar tooth extracted from human being in a dental clinic. At first, the collected tooth is pretreated by a supersonic tooth-cleaning machine in order to remove dental calculus and stain on the surface thereof. Next, a low speed saw is used to cut the side surface portion of the tooth crown (thickness of about 1˜2 mm). A high speed dental drill is used to grind the tooth. Several pieces of squared specimens with a side length of 0.5 cm are obtained. Finally, the specimens are placed in a supersonic tank to be cleaned for future use.

NaF is dissolved in deionized water and thus a 4000 ppm NaF solution is prepared. KH₂PO₄ and K₂HPO₄ with a molar ratio of 2:3 are added as the buffer composition and 5.0 g/100 ml H₂O of glycerin is added to inhibit water dissipation. The solution is then stirred until uniform and 3.0 g/100 ml H₂O of carbopol is added. The solution is stirred for 6˜8 hrs at a room temperature to become colloidal.

Waterproof handi-wrap is used as the support substrate for the strip. The preparation method of a fluoride-releasing strip for tooth is described in the following. The handi-wrap used as the support substrate is wrapped on a glass plate that is laminated with a release film. The prepared colloidal solution is coated on the handi-wrap by an adjustable blade to form a 500 μm film, according to the general thickness 200˜600 μm for a scraped film. Several blocks with a pre-measured area are cut by scissors and weighted by an electronic balance. Thus, the required quantity of the solution per unit area for forming the strip with an average thickness of 500 μm is calculated. Therefore, in the experimental operation, according to the surface area of the tooth specimen, the fluoride-containing solution is weighted and applied on the handi-wrap and then coated on the enamel surface of the tooth specimen.

Each tooth is divided into four pieces. The fluoride-containing solution is coated on the enamel surface of the tooth specimen, on which the strip is attached at 35° C. for 0 hr (control), 4 hrs, 8 hrs, and 24 hrs. The elemental analysis of the surface is done by scanning electron microscope (SEM)/energy dispersive X-ray spectrometer. The energy dispersive X-ray spectrometer utilizes a high energy electron beam to bombard the to-be-tested surface to have the inner-shell electrons of the element on the surface ionized and to have thereby the outer-shell electron jump in to an inner shell vacancy to release X-ray and then uses an X-ray detector to detect the X-ray emitted from the element on the bombarded surface so as to determine element species and the relative ratio between elements according to the energy intensity of the detected X-ray. Therefore, crack or damage and uneven curved surface are tended to result in the errors on the measurement and analysis. Thus, each specimen has been enlarged 100 times to have five perfect blocks on the surface thereof for elemental analysis so as to prevent the errors on data due to the surface imperfection while using low magnification (×40). Data are selected according to the relative ratio between Ca and P in the elemental analysis. The major composition of tooth enamel is Ca₁₀(PO₄)₆(OH)₂ and thus the theoretical relative ratio between Ca and P (Ca/P ratio) is 1.67. Although the Ca/P ratio may be different for different specimens and may have some variation due to measurement errors, generally Ca/P ratio=1.5˜2.0 is used as the approximate standard. Therefore, if there is a large error in the Ca/P ratio due to the defect on the surface of the specimen, the corresponding data will be deleted.

The characterization of the surface components is done by Raman spectrometer and Secondary Ion Mass Spectrometry. Raman spectra are acquired by irradiating a sample with a powerful laser source of visible or near-IR monochromatic radiation. Secondary Ion Mass Spectrometry can separate the ions on the basis of their mass-to-charge ratio (m/z), where m is the mass number of the ion in atomic mass units and z is the number of fundamental charges that it bears. Both instruments are versatile and widely used tools for identifying the elements present in samples of matter.

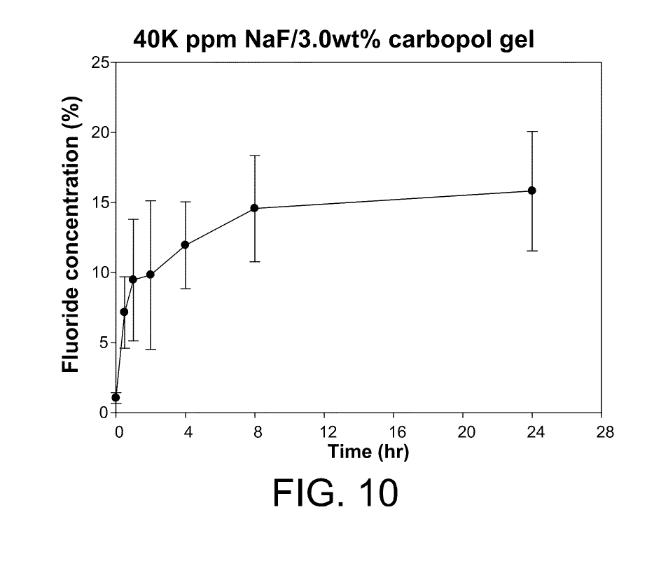

As shown in FIG. 10, the fluoride concentration of the tooth enamel surface is saturated after the strip is attached for 8 hrs, this is, the fluoride concentration of the tooth surface can reach the maximum value for 8 hrs of strip attaching. According the report, in the case of average bed time of 8 hrs and 40 minutes, utilizing the strip according to the invention during bed time can make the strip have the best fluoride releasing effect and additionally can prevent bad appearance while using in day time and strip ablation while eating.

After applied high-concentrated fluoride, the enamel surface of the tooth has calcium fluoride formed thereon which is an important medium for dental caries resistance. Fluoride-ions are released because of gradual dissolution of CaF₂ to have higher fluoride-ion concentration around the tooth surface so as to continuously inhibit tooth demineralization and promote remineralization. Therefore, after using fluoride compound, the precipitation capability and quantity of CaF₂ on the tooth surface are directly related to the acid resistance of the tooth. However, calcium fluoride precipitated on the sound enamel surface is easy to fall off by tiny physical force due to lack of chemical bonding with the tooth surface. On the contrary, if the tooth surface is eroded by acids to have micropores partially, calcium fluoride can precipitate on a larger surface area and the calcium fluorides formed in pores are not easily disturbed by external force to result in falling off. Therefore, better acid resistance can be obtained.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A fluoride-releasing strip for caries prevention, comprising:
a colloidal fluoride-containing solution, wherein said colloidal fluoride-containing solution comprises: a fluoride solution, at least one buffer, at least one moisturizer for inhibiting water dissipation and a carboxymethyl cellulose (CMC), and the concentration of fluoride ions in said colloidal fluoride-containing solution ranges from 2500 ppm to 4600 ppm; and
a support substrate, wherein said colloidal fluoride-containing solution is applied on said support substrate;
wherein said fluoride-releasing strip for caries prevention is contacted with tooth for more than or equal to 6 hours to form calcium fluoride (CaF₂) on the tooth.

2. The strip according to claim 1, wherein said fluoride solution is sodium fluoride (NaF) solution.

3. The strip according to claim 1, wherein the demineralization of the tooth contacting with said fluoride-releasing strip for 6 hours is less than 0.7 mg/cm².

4. The strip according to claim 1, wherein the pH value of said colloidal fluoride-containing solution is about 6.2 to 6.5.

5. The strip according to claim 1, wherein the pH value of said colloidal fluoride-containing solution is about 4 to 5.

6. The strip according to claim 1, wherein said buffer comprises one compound selected from the group consisting of the following or any combination thereof: phosphates, nitrates, acetates, nitrites, borates, and oxalates.

7. The strip according to claim 1, wherein said moisturizer comprises one compound selected from the group consisting of the following or any combination thereof: glycerin, butylene glycol, hyaluronic acid, ceramides, hyaluronic acid, and collagen.

8. The strip according to claim 1, wherein the concentration of said moisturizer is from 3.0 g/100 ml H₂O to 5.0 g/100 ml H₂O.

9. The strip according to claim 1, wherein the concentration of said carboxymethyl cellulose is from 2 to 2.5 wt %.

10. A fluoride-releasing strip for caries prevention, comprising:
a colloidal fluoride-containing solution, wherein said colloidal fluoride-containing solution comprises: a fluoride solution, at least one buffer, at least one moisturizer for inhibiting water dissipation and a carboxymethyl cellulose (CMC), and the concentration of fluoride ions in said colloidal fluoride-containing solution ranges from 2500 ppm to 4600 ppm; and
a support substrate, wherein said colloidal fluoride-containing solution is applied on said support substrate;
wherein said fluoride-releasing strip for caries prevention is contacted with tooth to form calcium fluoride (CaF₂) on the tooth.

11. The strip according to claim 10, wherein said fluoride solution is sodium fluoride (NaF) solution.

12. The strip according to claim 10, wherein the demineralization of the tooth contacting with said fluoride-releasing strip for 6 hours is less than 0.7 mg/cm².

13. The strip according to claim 10, wherein the pH value of said colloidal fluoride-containing solution is about 6.2 to 6.5.

14. The strip according to claim 10, wherein the pH value of said colloidal fluoride-containing solution is about 4 to 5.

15. The strip according to claim 10, wherein said buffer comprises one compound selected from the group consisting of the following or any combination thereof: phosphates, nitrates, acetates, nitrites, borates, and oxalates.

16. The strip according to claim 10, wherein said moisturizer comprises one compound selected from the group consisting of the following or any combination thereof: glycerin, butylene glycol, hyaluronic acid, ceramides, hyaluronic acid, and collagen.

17. The strip according to claim 10, wherein the concentration of said moisturizer is from 3.0 g/100 ml H₂O to 5.0 g/100 ml H₂O.

18. The strip according to claim 10, wherein the concentration of said carboxymethyl cellulose is from 2 to 2.5 wt %.
